(19) United States
(12) Patent Application Publication
(10) Pub. No.: US 2009/0136545 A1
(43) Pub. Date: May 28, 2009

(54) PORCINE ROTAVIRUS REASSORTANT COMPOSITIONS

(76) Inventors: Yasutaka Hoshino, Wheaton, MD (US); Albert Z. Kapikian, Rockville, MD (US)

Correspondence Address: TOWNSEND AND TOWNSEND AND CREW, LLP, TWO EMBARCADERO CENTER, 8TH FLOOR, SAN FRANCISCO, CA, 94111, US

(21) Appl. No.: 11/994,645

(22) PCT Filed: Jul. 10, 2006
(86) PCT No.: PCT/US2006/026901

§ 371 Date: Jul. 14, 2008

Related U.S. Application Data
(60) Provisional application No. 60/698,572, filed on Jul. 11, 2005.

(51) Int. Cl. A61K 39/15

(52) U.S. Cl. 424/215.1

(57) ABSTRACT

The present invention provides rotavirus reassortant immunogenic compositions based on a porcine rotavirus. In particular, porcine rotavirus Gottfried strain-based single VP7 or VP4 gene substitutions which can provide (i) an attenuation phenotype of a porcine rotavirus in humans and (ii) antigenic coverage for G serotypes 1, 2, 3, 4, 5, 6, 8, 9 and 10 and P serotype 1A[8], 1B[4] and 2A[6]. The compositions have been demonstrated to induce consistent levels of neutralizing antibodies against rotavirus specificities which are of global epidemiologic importance. Porcine rotavirus-based reassortant rotavirus compositions induce neutralizing antibodies against P1A[8] and P2A[6] VP4 serotypes which may provide an advantage over rhesus- or bovine-based reassortant vaccines since the VP4s of the latter vaccines do not evoke antibodies capable of neutralizing the P1A[8], or P2A[6] VP4.

### CROSS-REFERENCES TO RELATED APPLICATIONS

The present application claims priority to U.S. Provisional Patent Application No. 60/698,572, filed Jul. 11, 2005, the entire disclosure of which is incorporated by reference herein.

### BACKGROUND OF THE INVENTION

Rotavirus diarrhea poses an immense health burden in both developing and developed countries. It is estimated to be responsible for up to 592,000 deaths annually in children less than 5 years old, predominantly in developing countries (Parashar et al., Emerg. Infect. Dis. 2003, 9:565-572). Although deaths from rotavirus infection occur much less frequently in developed countries, the overall health burden of rotavirus disease is enormous. In the United States alone, rotavirus infection has been estimated to cause each year approximately 20 deaths, 50,000 hospitalizations, 500,000 physician visits and more than US $1 billion in societal costs (Bresse et al., Vaccine 1999, 17:2207-2222; Glass et al, Science 1994, 265:1389-1391; Kapikian et al., Fields Virology, 4th ed., 1787-1833, 2001). Thus, the availability of a safe and effective rotavirus vaccine capable of preventing this enormous health burden would represent a global public health breakthrough. Rotavirus candidate vaccines that have been developed thus far by us and others are characteristically designed to be administered orally. This is because active immunity following either natural (Bishop et al., New Engl. J. Med. 1983, 309:72-76; Fischer et al., J. Infect. Dis., 2002, 186:593-597; Velazquez et al., New Engl. J. Med., 1996, 335:1002-1008) or experimental oral (Wyatt et al., Science, 1979, 203:548-550; Hoshino et al., J. Virol., 1988, 62:744-748; Offit et al., J. Virol., 1986, 60:491-496) rotavirus infection has been demonstrated to provide protection against subsequent rotavirus disease. Such candidate vaccines include a rhesus monkey rotavirus (RRV)-based quadrivalent vaccine (RotaShield™, Wyeth-Lederle Vaccines and Pediatrics, USA; BIOVIRx, USA) (Advisory Committee on Immunization Practices, MMWR Morb Mortal Weekly Rep, 1999, 53:949-954; Midthun et al., J. Virol., 1985, 53:949-954; Midthun et al., J. Clin. Microbiol., 1986, 24:822-826; Spiegel et al., Vaccines Against Rotavirus Disease, Federal Register 2003, 68:7576.7), bovine rotavirus (UK)-based tetravalent vaccine (National Institutes of Health, USA) (Midthun et al, J. Virol., 1985, 53:949-954; Midthun et al., J. Clin. Microbiol., 1986, 24:822-826), bovine rotavirus (WC3)-based pentavalent vaccine (Rotateq™, Merck, USA) (Offit, Seminars Pedatr. Infect. Dis., 2002, 13:190-195), monovalent human rotavirus vaccine Rotarix™, GlaxoSmithKline, Belgium) (Ward, Viral Immunol., 2003, 16:17-24), monovalent human rotavirus (RC3) vaccine (University of Melbourne, Australia) (Barnes et al., Vaccine, 2002, 20:2950-2956), monovalent human rotavirus (116E; I-321) vaccine (Bharat Biotech Ltd, India) (Cunliffe et al, J. Infect., 2002, 45:1-9; Glass et al., Lancet, 2004, 363:1547-1550) and nonovalent lamb rotavirus vaccine (LLR, Lanzhou Institute of Biological Products, China) (Cunliffe et al., J. Infect., 2002, 45:1-9; Glass et al., Lancet, 2004, 363:1547-1550). Such candidate rotavirus vaccines have been or are being evaluated clinically in different populations in various parts of the world (for reviews, see Cunliffe et al., J. Infect., 2002, 45:1-9; Glass et al., Lancet, 2004, 363:1547-1550; Offit et al., in, Viral Gastroenteritis, Amsterdam, Elsevier, pgs. 345-356, 2003).

Rotavirus outer capsid proteins VP7 and VP4 have been well established to be capable of inducing independent neutralizing antibodies (Greenberg et al, J. Virol., 1983, 47:267-275; Hoshino et al., Proc. Natl. Acad. Sci. USA, 1985, 82:8701-8704; Offit et al., J. Virol., 1986, 57:376-378) and associated protection against disease (Hoshino et al., J. Virol., 1988, 62:744-748; Offit et al., J. Virol., 1986, 60:491-496; Matsui et al., Adv. Virus Res., 1989, 36:181-214). Thus a binary system for classification and nomenclature of two neutralization specificities of rotavirus has been established: VP7 or G (VP7 is a glycoprotein) serotype and VP4 or P (VP4 is protease sensitive) serotype (Kapikian et al., Fields Virology, 4th ed., 1787-1833, 2001; Estes, Fields Virology, 4th ed., 1747-1785, 2001). Since neutralizing antibodies and their type specificity have been reported to play an important role in protection against many viral diseases, most rotavirus candidate vaccines were generated in consideration of G and/or P types of epidemiologic importance. For example, (i) rhesus rotavirus-based and bovine rotavirus-based multivalent vaccines have been designed to provide antigenic coverage for globally important G (i.e., G1-G4 and G9) and P (i.e., 1A[8] or 1B[4]) serotypes (Midthun et al., J. Virol., 1985, 53:949-954; Midthun et al., J. Clin. Microbiol., 1986, 24:822-826; Offit, Seminars Pedatr. Infect. Dis., 2002, 13:190-195; Hoshino et al., Vaccine, 2002, 20:3576-3584; Hoshino et al., Vaccine, 2003, 21:3003-3010), (ii) monovalent human rotavirus vaccine (Rotarix™) is designed to cover the most globally common G-P combination (G1 and P1A) (Ward, Viral Immunol., 2003, 16:17-24) and (iii) monovalent 116E and 1321 vaccines should provide antigenic coverage for regionally important G serotypes (G9 and G10 in India) (Cunliffe et al., J. Infect., 2002, 45:1-9; Glass et al., Lancet, 2004, 363:1547-1550).

Approximately two hundred years ago, Jenner pioneered the use of a naturally occurring animal pathogen to immunize humans against the human pathogen counterpart to control smallpox (Baxby, Vaccine, 1999, 17:301-307). In the Jennerian approach to vaccine candidate development, an antigenically related animal virus that is restricted in replication and attenuated in humans is utilized as a vaccine to protect against the related human disease. The Jennerian and modified Jennerian approaches have been successfully employed to develop various rotavirus candidate vaccines that include rhesus rotavirus-based and bovine-rotavirus-based multivalent vaccines listed earlier (Midthun et al., J. Virol., 1985, 53:949-954; Midthun et al., J. Clin. Microbiol., 1986, 24:822-826; Offit, Seminars Pedatr. Infect. Dis., 13:190-195, 2002; Hoshino et al., Vaccine, 20:3576-3584, 2002; Hoshino et al., Vaccine, 2003, 21:3003-3010). Such vaccines are typically attenuated in humans due to host range restriction but possess the protective antigens of the human virus and have been demonstrated to be highly effective in protection against severe rotavirus diarrheal illness in infants and young children (for reviews, see (Kapikian et al., Fields Virology, 4th ed., 1787-1833, 2001)). One of the characteristics of such rhesus- or bovine-based reassortant vaccines is that the VP4 of simian and bovine rotaviruses employed to construct the vaccines is distinct serotypically from human rotavirus VP4s of epidemiologic importance (Hoshino et al., Vaccine, 2002, 20:3576-3584).

Porcine rotaviruses are unique among mammalian and avian rotaviruses because of their relationship to human rotaviruses. For example, (i) porcine rotaviruses are closely related genetically to human rotaviruses as determined by RNA-RNA hybridization (Flores, Arch. Virol., 1986, 87:273-285), (ii) eight of the 10 G (VP7) serotypes described in humans have been detected in pigs, whereas, in contrast, only four and one of the 10 human rotavirus G serotypes have been detected in cows and rhesus monkeys, respectively (Kapikian et al., Fields Virology, 4th ed., 1787-1833, 2001; Liprandi et al., Virology, 2003, 315:373-380; Martella et al., Clin. Diagn. Lab. Immunol., 2001, 8:129-132), and (iii) four of the 10 P (VP4) serotypes or genotypes detected in humans have been shown to exist in pigs whereas, in contrast, only two and none of the 10 human rotavirus P types have been detected in cows and rhesus monkeys, respectively (Kapikian et al., Fields Virology, 4th ed., 1787-1833, 2001; Liprandi et al., Virology, 2003, 315:373-380; Martella et al., Clin. Diagn. Lab. Immunol., 2001, 8:129-132). In addition, human rotavirus bearing a P1A[8]G1 specificity (the predominant P-G combination detected in diarrheal patients worldwide) can not only infect piglets but also cause diarrhea in such animals and the gnotobiotic piglet model has extensively been utilized in studies of rotavirus pathogenesis, immunity and vaccine evaluation (for reviews, see (Yuan et al., Vet Immunol. Immunopathol., 2002, 87:147-160). Curiously, however, the Jennerian or modified Jennerian approach to rotavirus vaccine development has not been applied to porcine rotaviruses.

Rotavirus strains bearing the VP4 gene P[6] allele in conjunction with various G types, which were detected initially in characteristically symptom-free or only minimally symptomatic newborn babies, have recently been detected in an increasing number of infants and young children with diarrhea throughout the world (for reviews, see (Hoshino et al., Virology, 2003, 316:1-8; Santos et al., Rev. Med. Virol., 2005, 15:29-56), and references therein). For example, a recent strain survey involving infants and young children with rotavirus diarrhea in 14 countries in Africa reported that during 1996 to 1999 the predominant strains circulating across this continent bore a P[6] specificity in association with G1 or G3 (Steele et al., Vaccine, 2003, 21:361-367). It has been previously shown (Gorziglia et al., J. Virol., 1990, 64:414-418; Li et al., J. Clin. Micrbiol., 1993, 31:3075-3077) and recently confirmed (Hoshino et al., Virology, 2003, 316:1-8) that the VP4 (P2B[6]) of porcine rotavirus Gottfried strain isolated in the United States was closely related serotypically to human rotavirus VP4 with a P2A[6] specificity.

The present invention provides methods for the construction, characterization and administration of porcine rotavirus Gottfried-based immunogenic compositions which can provide (i) an attenuation phenotype of a porcine rotavirus in humans and (ii) antigenic coverage for serotypes G1, G2, G3, G4, G5, G6, G8, G9 and G10 as well as for P1A[8], P1B[4] and P2A[6]. The porcine rotavirus Gottfried-based reassortants are also shown to be protective in pigs against human rotaviral infection.

### BRIEF SUMMARY OF THE INVENTION

The present invention provides immunogenic reassortant rotavirus compositions and methods for their use in humans. The compositions described herein are produced by combining monovalent reassortant porcine rotaviruses with a human rotavirus, or other animal virus with antigens immunologically cross-reactive with a human rotavirus serotype, so as to provide one of each of the most clinically relevant serotypes of group A human rotavirus in a formulation which induces an at least partially protective human rotavirus-specific antibody response.

The immunogenic compositions of the invention specifically comprise a combination of reassortant porcine rotaviruses and a physiologically acceptable carrier to form a multivalent composition. In a particular embodiment, the multivalent immunogenic composition comprises a combination of reassortant porcine rotaviruses of the clinically relevant serotypes of human rotavirus that are most prevalent in various regions of the world, to form immunogenic compositions. The immunogenic compositions can be administered to an individual in need of immunological protection against rotavirus, such as, e.g., an infant, child or adult in an amount sufficient to induce an immunogenic response specific to the human rotavirus serotype. The compositions elicit the production of an immune response that is at least partially protective against symptoms of serious rotaviral disease, such as severe diarrhea and dehydration, when the individual is subsequently infected with a wild-type human rotavirus strain.

Certain of the compositions of the present invention take advantage of the unique characteristics of the porcine rotavirus Gottfried strain. In particular, the porcine rotavirus Gottfried strain is a serotype (P[6],G4). The VP7 G4 serotype represents one of four human rotavirus G serotypes that are of global epidemiologic importance as compared to another important reassortant rotavirus immunogenic composition comprising the bovine rotavirus UK strain which is (P[5],G6). Also, the porcine rotavirus VP4 neutralization specificity (P[6]) is closely related to that of human rotavirus strains with P[6] specificity that are of clinical importance. Further, the porcine rotavirus Gottfried strain gene sequences are, as determined by RNA-RNA hybridization, closely related to those of human rotavirus. Each of these characteristics make immunogenic compositions comprising porcine Gottfried rotavirus x rotavirus that encode antigens immunologically cross-reactive with human rotavirus antigens having VP7 serotypes 1, 2, 3, 4, 5, 6, 8, 9, 10 and/or VP4 serotype 1A and 1B particularly useful.

### BRIEF DESCRIPTION OF THE DRAWINGS

FIG. 1 shows electrophoretic migration patterns of genomic RNAs of human rotavirus D strain (lane 1), reassortant Gott x D (lane 2), porcine rotavirus Gottfried strain (lane 3), reassortant Gott x DS-1 (lane 2), porcine rotavirus Gottfried strain (lane 3), reassortant Gott x DS-1 (lane 4), human rotavirus DS-1 strain (lane 5), human rotavirus M strain (lane 6), reassortant Gott x M (lane 7), porcine rotavirus Gottfried strain (lane 8), reassortant Gott x ST3 (lane 9), human rotavirus ST3 strain (lane 10), human rotavirus IAL28 strain (lane 11), reassortant Gott x IAL28 (lane 12), and porcine rotavirus Gottfried strain (lane 13) in 10% polyacrylamide gel. Genomic RNAs were electrophoresed at 13 mA for 15 hours and the resulting migration patterns were visualized by staining of gel with silver nitrate.

FIG. 2 shows an electrophoretic migration pattern of genomic RNAs of bovine rotavirus UK strain (lane 1), reassortant Gott x UK (lane 2), porcine rotavirus Gottfried strain (lane 3), reassortant Gott x 1290 (lane 4), human rotavirus 1290 strain (lane 5), human rotavirus AU32 strain (lane 6), reassortant Gott x AU32 (lane 7), porcine rotavirus Gottfried strain (lane 8), reassortant Gott x KC-1 (lane 9), bovine rotavirus KC-1 strain (lane 10), human rotavirus Mo strain (lane 11), reassortant Mo x Gott (lane 12), porcine rotavirus Gottfried strain (lane 13), reassortant DS-1 x Gott (lane 14), and human rotavirus DS-1 strain (lane 15) in 10% polyacrylamide gel. Genomic RNAs were electrophoresed at 13 mA for 15 hours and the resulting migration patterns were visualized by straining of gel with silver nitrate.

### DETAILED DESCRIPTION OF THE INVENTION

The present invention provides immunogenic reassortant rotavirus compositions for use in humans. The compositions described herein are produced by combining monovalent reassortant animal rotaviruses with a human rotavirus, or other animal virus with antigens immunologically cross-reactive with a human antigen, so as to provide one of each of the most clinically relevant serotypes of group A human rotavirus in a formulation which induces a human rotavirus-specific antibody response.

Thus, the immunogenic compositions of the invention specifically comprise a combination of reassortant porcine rotaviruses and a physiologically acceptable carrier to form a multivalent composition. In a particular embodiment, the multivalent immunogenic composition comprises a combination of reassortant porcine rotaviruses of the clinically relevant serotypes of human rotavirus that are most prevalent in various regions of the world, to form immunogenic compositions. The immunogenic compositions can be administered to an individual in need of immunological protection against rotavirus, such as, e.g., an infant, child or adult in an amount sufficient to induce an immunogenic response specific to the human rotavirus serotype. The compositions elicits the production of an immune response that is at least partially protective against symptoms of serious rotaviral disease, such as severe diarrhea and dehydration, when the individual is subsequently infected with a wild-type human rotavirus strain. This reactivity has been demonstrated in an animal model. As the reassortant viruses of the immunogenic composition infect the host alimentary tract, some mild disease may occur as a result of the vaccination, but typically the immunogenic composition of the present invention will not cause clinically relevant fever or reaction in the vaccine. Following vaccination, there are detectable levels of host engendered serum antibodies which are capable of neutralizing the serotypes of rotavirus that make up the immunogenic composition. In particular, the multivalent immunogenic composition of the present invention will produce an effective immunological response to most, if not all, of the clinically relevant group A human rotaviruses prevalent in each different selected setting. The teachings of the present invention are not limited to those human rotavirus serotypes currently recognized as clinically relevant, but also include those serotypes of human rotavirus that emerge as clinically relevant in the future.

The reassortant rotavirus which is a component of the immunogenic composition of the present invention is in an isolated and typically purified form. By isolated is meant to refer to reassorted rotavirus that has been separated from other cellular and viral products of its manufacture, such as wild type virus, or parental strain rotavirus, and other heterologous components of a cell culture or other systems.

Generally, rotavirus reassortants are produced by co-infection of mammalian cells in culture with a tissue culture-adapted animal rotavirus, i.e., porcine, bovine, rhesus, and the like, and a tissue culture-adapted human rotavirus. Typically, African green monkey kidney (AGMK) cells are used as the host cells for co-infection. Following co-infection with the animal and human rotavirus strains, selection of the desired reassortant is typically achieved by exposing the growth yield of co-infected cultures to neutralizing antibodies specific for the protein product of the animal rotavirus gene that is to be replaced by the human rotavirus gene (See, U.S. Pat. No. 4,571,385, incorporated herein by reference). In particular, polyclonal serum or monoclonal antibody specific for porcine rotavirus VP7 and/or VP4 proteins can be used. After several rounds of plaque purification and subculture, selected reassortants are characterized for serotype and genotype. Serotype is typically determined by plaque reduction neutralization (PRN) assay or enzyme immunoassay. Genotype is typically determined by gel electrophoresis and RNA-RNA hybridization of the viral genome. Rotavirus reassortants having only the human VP7 or VP4 gene are typically selected for the present multivalent immunogenic compositions. Reassortants comprising multiple human rotavirus genes can also be used. In this regard, reassortant rotaviruses of interest are particularly those encoding the human rotavirus VP7 and/or the human rotavirus VP4 gene products. In particular, porcine x human reassortant rotavirus having human rotavirus VP4 (P1A, P1B and P2A) single gene reassortants and VP7 (G1, G2, G3, G4, G5, G6, G8, G9 and G10) single gene reassortants are disclosed.

In the present invention, particularly preferred rotavirus reassortants are human rotavirus and porcine rotavirus reassortants comprising the human rotavirus gene encoding VP7 and the remaining ten rotavirus genes of porcine rotavirus origin. The porcine rotavirus strain Gottfried (Bohl et al., J. Clin. Microbiol., 1984, 19:105-111) is particularly preferred because of its pedigree and its immunological similarity to certain human rotavirus serotypes. The close serotypic relatedness to human rotavirus VP4 with a P2A[6] specificity could theoretically make the Gottfried VP4 protective against human rotaviruses bearing P2A[6] specificity. Furthermore, it has been shown that following hyperimmunization of guinea pigs, the Gottfried VP4 induced low but consistent levels of antibodies capable of neutralizing selected human rotaviruses bearing the globally important P1A[8] or P1B[4]. Certain porcine reassortants with other animal VP7 or VP4 genes can be used to induce an immune response in a human that is serologically cross-reactive with certain human VP7 and/or VP4 serotypes. For example, the bovine KC-1 VP7 gene has been shown to express a protein which is serologically cross-reactive with human VP7 G10.

Human rotavirus can be isolated for use in making the immunogenic compositions of the present invention. In the particular embodiments of the present invention human rotavirus D strain (P1A[8], G1), M strain (P[8], G3), ST3 (P2A[6], G4), Mo (P1A[8], G1), DS-1 (P1B[4], G2, IAL28 (P1A[8], G5), 1290 (P[4], G8), AU32 (P1A[8], G9) have been used to construct porcine x human reassortant rotavirus. To obtain reactivity to the human VP7 G10 serotype a bovine rotavirus, KC-1 (P8[11], G10) with a VP7 protein cross-reactive with the human VP7 G10 serotype has been used to construct a porcine x bovine reassortant rotavirus. Further, to obtain reactivity to the human VP7 G6 serotype, a bovine rotavirus, UK (P7[5], G6) with a VP7 protein cross-reactive with the human VP7 G6 serotype has been used to construct a porcine x bovine reassortant rotavirus.

In an alternative embodiment, reassortant rotavirus of a specific serotype can be produced using a previously obtained reassortant. For example, to produce additional porcine Gottfried reassortants the porcine Gottfried reassortant comprising the human rotavirus VP7 serotype D strain Gott x D (P2B[6],G1) can be used to produce porcine Gottfried rotavirus reassortants having VP7 antigens immunologically cross-reactive with human VP7 serotypes of G2, G3, G4, G5, G6, G8, G9, or G10. The methods used are similar to those described above except polyclonal or monoclonal neutralizing antibody specific for the VP7 serotype of the parental human rotavirus reassortant is used to select for new reassortants of the desired human, bovine, and/or porcine rotavirus VP7 serotype. It is also contemplated as part of the present invention that as other clinically relevant human VP4 or VP7 serotypes are isolated and identified reassortant rotavirus of the newly discovered serotype can be produced by the described methods.

Propagation of the reassorted rotavirus can be in a number of cell cultures which support rotavirus growth. Preferred cell cultures for propagation of rotavirus reassortants for vaccine use include primary or secondary simian African green monkey kidney cells (AGMK), qualified diploid simian FRhL-2 cells and qualified simian heteroploid Vero cells. Cells are typically inoculated with rotavirus reassortants at a multiplicity of infection ranging from about 0.1 to 1.0 per cell, or more, and are cultivated under conditions appropriate for viral replication, for about 3 to about 5 days, or as long as necessary for virus to reach an adequate titer. Rotavirus reassortants are harvested from infected cell culture and separated from cellular components, typically by well known clarification procedures, e.g., centrifugation, and may be purified as desired using procedures well known to those skilled in the art.

In a typical embodiment for use as an immunogenic composition, a porcine x human reassortant rotavirus of serotype 1, serotype 2, serotype 3, and serotype 4 are used as a multivalent vaccine. Typically, the porcine x human reassortant rotavirus or porcine x bovine reassortant rotavirus of each of the serotypes will be admixed to form a combined composition for simultaneous administration. The final ratio of each rotavirus serotype is determined by the immunogenicity of the individual rotavirus reassortants. In additional embodiments, porcine reassortants having a VP7 antigen cross-reactive with human VP7 serotypes of G5, G6, G8, G9 and/or G10 can be used in any combination with the VP7 serotype G1, G2, G3, and G4 in a combined composition. These VP7 serotypes have been detected in an increasing number of patients with diarrhea in various parts of the world. Porcine x human reassortant rotavirus compositions comprising VP4 serotypes P1A and P1B can also be included in a combined composition. Although not preferred, each porcine rotavirus reassortant, or a combination thereof, can also be administered in a sequential manner to provide an effective vaccine formulation.

Porcine reassortant rotavirus multivalent immunogenic compositions of the present invention contain as an active ingredient an immunogenically effective amount of each of at least the clinically most important VP7 serotypes of human rotavirus as described herein. In particular, each antigenically distinct porcine x human reassortant rotavirus is administered at a dosage of about 10^{4.0} plaque forming units to about 10¹⁰ plaque forming units. The immunogenic composition can be introduced into a host, particularly a human, with a physiologically acceptable carrier and/or adjuvant. Useful carriers include, e.g., citrate-bicarbonate buffer, buffered water, normal saline, and the like. The resulting aqueous solutions may be packaged for use as is, or lyophilized, as desired, using lyophilization protocols well known to the artisan. Lyophilized virus will typically be maintained at about 4° C. When ready for use the lyophilized preparation is combined with a sterile solution prior to administration, as mentioned above.

The compositions can contain pharmaceutically acceptable auxiliary substances as required to approximate physiological conditions, such as pH adjusting and buffering agents and the like, for example, sodium acetate, sodium lactate, sodium chloride, potassium chloride, calcium chloride, sorbitan monolaurate, tri-ethanolamine oleate, citrate-bicarbonate, or the like. When the composition is administered orally it may also be necessary to provide the individual a buffer solution to partially neutralize stomach acid and protect the reassortant rotavirus while passing to the intestine. Buffer solutions appropriate for this use include sodium bicarbonate, citrate bicarbonate, or the like. Upon immunization with a porcine x human reassortant rotavirus composition of the present invention, particularly via the oral route, the immune system of the host responds to the composition by producing both local secretory and serum antibodies specific for the rotavirus proteins. As a result of the administration of the composition, the host becomes at least partially or completely immune to human rotavirus disease caused by a wild-type strain that corresponds to the immunizing serotype(s). If wild-type virus infection does occur, the host is resistant to developing moderate or severe rotaviral disease, particularly of the gastrointestinal tract.

Compositions of the present invention are tested for safety and initial effectiveness by administration to various animal models. The compositions of the present invention in particular can be tested in the gnotobiotic pig model. In this model it has been determined that human rotavirus bearing a P1A [8], G1 specificity not only infect piglets but also cause diarrhea in the animals. This provides an opportunity to test the reassortant porcine x human compositions of the invention for not only immunogenicity in the piglet model, but also to test for protection against an infecting human rotavirus strain.

The gnotobiotic pig model has several advantages over other animal models for studies of passive and active immunity to human rotavirus. In comparison with mice and calves, the intestinal tract physiology, body size, type of passive immunity (milk IgA) and immune development of piglets resemble those of human infants more closely. As above, pigs are susceptible to human rotavirus infection and produce subsequent diarrhea. The gnotobiotic environment prevents natural exposure to rotavirus or other diarrheal pathogens that could confound data analysis. In addition, pigs are born devoid of maternal antibodies, acquiring serum Igs by intestinal absorption of colostrum Igs for up to 36 hrs after birth and before gut closure (cessation of absorption of intact Ig) occurs. This facilitates experimental manipulation of levels of passive antibodies. The gnotobiotic pig model is particularly useful for testing the immunogenicity of the compositions of the present invention because the reassortant viruses will induce an immune response that can kill infecting human rotavirus.

The multivalent immunogenic compositions of the present invention containing the porcine x human reassortant rotaviruses are administered to animals and to a person, particularly an infant, susceptible to or otherwise at risk of rotavirus disease to induce the individual's own immune response capabilities. Such an amount is defined to be an “immunogenically effective dose.” Immunogenicity or “immunogenically effective dose” as used in the present invention means the development in a vaccine of a cellular and/or antibody mediated immune response to the vaccine composition. Usually such a response consists of the vaccine producing serum antibodies, B cells, helper T cells, suppressor T cells, and/or cytotoxic T cells directed specifically to an antigen or antigens included in the vaccine composition of the present invention. A four-fold or greater rise above a pre-inoculation antibody titer following immunization measured by a rotavirus group-specific, or rotavirus serotype-specific assay is considered a significant response.

In this use, the precise amount of each porcine x human reassortant rotaviral serotype in a particular immunogenic composition depends on the patient's age, state of health and weight, the mode of administration, the nature of the formulation, and the like, but generally the range was from about 10⁴ to about 10¹⁰ plaque forming units, preferably from about 10⁵ to less than 10⁸ plaque forming units (pfu) of each serotype per patient.

In any event, the formulations for the immunogenic composition should provide a quantity of each porcine x human reassortant rotavirus of the invention sufficient to induce an individual's immune response against rotavirus disease.

In some instances it may be advantageous to combine a multivalent porcine x human reassortant rotaviral composition of the present invention with other serotypes of human rotavirus or other infectious agents, particularly, other gastrointestinal viruses. For example, the multivalent porcine x human reassortant rotaviral composition having porcine x human reassortant rotavirus of VP7 serotypes 1, 2, 3, and 4 can further include, for example, porcine x human reassortant rotavirus of serotype 5 (Timenetsky et al., J. General Virol. 1997, 78:1373-1378), and/or serotype 9 (Nakagomi et al., Microbiol. Immunol., 1990, 34:77-82), and/or porcine x bovine reassortant rotavirus which is cross reactive with human rotavirus serotype 10 and/or human x porcine reassortant rotavirus of VP4 serotype 1A and/or 1B. Administration can be simultaneous (but typically separately) or sequentially with another possible gastrointestinal virus vaccine, such as a human calicivirus (e.g., Norwalk virus) or related vaccine.

Single or multiple administrations of the immunogenic compositions of the invention can be carried out. In neonates and infants, multiple administrations may be required to elicit a sufficient level of immunity, particularly where there are high levels of maternally derived antibodies specific for rotavirus. Administration can begin within the first 2 to 4 months of life, and continue at intervals such as one to two months or more after the initial immunization, or as necessary to induce and maintain sufficient levels of immunity against human rotavirus infection. Administration can also be within the first four weeks of life to avoid the natural window of incidence of intussusception. In this administration schedule a second dose is typically provided no sooner then three weeks later, but not later than about 8 weeks. No administration should be provided after 8 weeks of life. Adults who are particularly susceptible to repeated or serious rotavirus disease, such as, for example, health care workers, day care workers, family members of young children, the elderly, and the like may require multiple immunizations to establish and/or maintain an effective immune status. Levels of induced immunity can be monitored by measuring amounts of rotavirus group-specific antibodies or serotype-specific neutralizing antibodies in serum and secretions, and dosages adjusted or vaccinations repeated with one or more serotypes of a multivalent reassortant rotavirus composition of the present invention when necessary to maintain desired levels of immunity.

The following examples are offered by way of illustration, not by way of limitation.

### EXAMPLES

In the following example various porcine rotavirus reassortants are constructed and characterized. The porcine rotavirus reassortants can be combined to form immunogenic compositions for administration to individuals to induce a VP7 and/or VP4 specific immune response that can be useful in protecting the individual from infection by a wild-type human rotavirus.

### Materials and Methods

### Viruses, Cell Cultures, Culture Medium, Virus Titration, Neutralization Assay and Hyperimmune Antiserum:

A stool sample containing porcine rotavirus Gottfried strain (P2B[6],G4) (Bohl et al., J. Clin. Microbiol., 1984, 19:105-111) which was derived from an infected gnotobiotic piglet was a gift from Dr. Edward Bohl (Ohio State University, Wooster, Ohio). This virus was adapted to growth in primary or secondary African green monkey kidney (AGMK) cells (Diagnostic Hybrids, Athens, Ohio) and passaged 11 times including a triple plaque purification. A 10% suspension of a stool derived from a gnotobiotic calf infected with human rotavirus D strain (P1A[8],G1) (Wyatt et al., J. Clin. Micobiol., 1983, 18:310-317) or M strain (P[8],G3) (Wyatt et al., Infect. Immun., 1982, 37:110-115) was passaged 10 times and 12 times, respectively, in primary or secondary AGMK cell cultures. The origin and passage history of human rotavirus strains Mo (P1A[8],G1), DS-1 (P1B[4],G2), IAL28 (P1A[8],G5), 1290 (P[4],G8) and AU32 (P1A[8],G9) and bovine rotavirus strain UI (P7[5],G6) and KC-1 (P8[11],G10) were reported previously (Hoshino et al., Vaccine, 2002, 20:3576-3584; Hoshino et al., Vaccine, 2003, 21:3003-3010). Primary or secondary cultures of AGMK cells were used for genetic reassortment, plaque purification and virus amplification. The monkey kidney MA104 cell line was used for virus titration and plaque reduction neutralization (PRN) assays. Eagle's minimum essential medium supplemented with 0.5 μg/ml trypsin (Sigma γ-irradiated trypsin, Sigma Chemical, St. Louis, Mo.) and antibiotics was used as maintenance medium and Leibovitz L-15 medium supplemented with antibiotics was employed when making virus or serum dilutions. The PRN assay was performed by using 50 to 60 PFU per 250 μl of the virus as described previously (Hoshino et al., Arch. Virol., 1998, 143:1233-1244). Agarose (SeaKem ME; BMA, Rockland, Me.) was used as a solidifying reagent in the plaque assay. Hyperimmune antiserum to each of the reassortants was raised in specific-pathogen free guinea pigs (Charles River, Wilmington, Mass.) which were free of rotavirus-neutralizing antibodies (titer of less than 1:20 versus Gottfried virus) as determined by PRN assay. Guinea pigs have been an animal of choice in laboratories for rotavirus-specific serum production since; (i) they are a rare animal species that does not appear to undergo natural rotavirus infection. Antibodies to rotaviruses have not been detected in hundreds of guinea pigs used over the last 24 years; (ii) they are appropriate in size, providing approximately 10 to 16 mls of serum per animal whereas, in comparison, mice are too small and rabbits are too large in size. In addition, the latter two animal species undergo natural rotavirus infection; and (iii) they are docile and easy to handle. Preparation of rotavirus immunogenic compositions and procedures for intramuscular immunizations of guinea pigs were previously reported (Wyatt et al., Infect. Imnmun., 1982, 37:110-115; Hoshino et al., J. Virol., 2004, 78:7802-7804). Sera were inactivated before use by heating at 56° C. for 30 min.

### Construction, Identification, and Characterization of Single VP7 or VP4 Gene Substitution Rotavirus Reassortants:

Roller tube cultures of primary AGMK cells were co-infected at a multiplicity of infection (MOI) of approximately one with human rotavirus strain D, Mo, DS-1, M, IAL28, 1290, AU32 or bovine rotavirus strain UK or KC-1 and porcine rotavirus strain Gottfried (Gott). When approximately 75% of the infected cells exhibited cytopathic effects, the cultures were frozen and thawed once, and the lysate was plated onto secondary AGMK cells in a 6-well plate in the presence of (i) G4-specific VP7 neutralizing monoclonal antibody (N-mAb) ST-2G7 (Taniguchi et al., J. Infect. Dis., 1987, 155:1159-1166) for selection of the desired Gott x D (P2B[6],G1), Gott x DS-1 (P2B[6],G2), Gott x M (P2B[6],G3), Gott x IAL28 (P2B[6],G5), Gott x UK (P2B[6],G6), Gott x 1290 (P2B[6],G8), Gott x AU32 (P2B[6],G9) or Gott x KC-1 (P2B[6],G10), (ii) G1-specific-N-mAb 2C9 (Shaw et al., J. Clin. Microbiol., 1985, 22:286-291) for selection of the desired Mo x Gott (P1A[8],G4) or (iii) G2-specific neutralizing monoclonal antibody S2-2G10 (Taniguchi et al., J. Infect. Dis., 1987, 155:1159-1166) for selection of the desired DS-1 x Gott (P1B[4],G4). Each of the desired reassortants was selected and identified and then plaque purified three times. The desired Gott x D (P2B[6],G1) reassortant thus constructed and human rotavirus strain ST3 were then employed to co-infect roller tube cultures of primary AGMK cells at an MOI of approximately one. The infected cell culture lysate was plated onto secondary AGMK cells in a 6-well plate in the presence of N-mAb 2C9 for selection of the desired Gott x ST3 (P2B[6],G4). The origin of genes of each plaque-purified reassortant was identified by polyacrylamide gel electrophoresis (PAGE) of its genomic RNAs (Kalica et al., Virology, 1978, 87:247-255; Rodger and Holmes, J. Virol., 1979, 30:839-846; Jones et al., J. Clin. Virol., 2003, 26:347-354). The origin of certain genes which was not able to be determined with certainty by PAGE was studied further by constant denaturant gel electrophoresis (CDGE) as described previously (Jones et al., J. Clin. Virol., 2003, 26:347-354). Hyperimmune guinea pig antiserum to each reassortant was tested for antibodies to selected human and animal rotavirus strains by 60% PRN assay.

Analysis of Protective Efficacy Provided by Reassortant Porcine Rotavirus Against Challenge with Virulent Human Rotavirus:

Antibody responses to VP4 or VP7 and protective efficacy of porcine Gottfried-human reassortant immunogenic compositions are tested in the gnotobiotic pig model. Porcine Gottfried-human reassortants with G1:P2B[6] (Gott x D) or G3:P2B[6] (Gott x M) specificities are used to immunize the piglets and the animals are challenged with virulent human rotavirus strain Wa (G1:P[8]) of M (G3:P[8]) and observed for clinical signs and virus shedding as described below.

Hysterectomy-derived, near-term pigs are randomly assigned to six (6) groups and housed in sterile isolation units as described previously (Meyer et al. Appl. Microbiol. 1964, 12:295-300). Piglets are maintained under gnotobiotic conditions and fed a commercial modified infant formula as the basic diet and are free of maternal antibody and devoid of exposure to extraneous pathogens. All piglets are inoculated orally at 3 to 5 days of age with the first dose of vaccine (either Gott x D (G1:P2B[6]) or Gott x M (G3:P2B[6]), or placebo. A subset of piglets from each group are challenged orally with approximately 10⁶ ID₅₀ of virulent (i.e., gnotobiotic piglet-passaged) human rotavirus (Wa or M; See Table 1) at about three weeks after the first-inoculation to evaluate protection.

TABLE 1
Protective efficacy provided by reassortant Gott × D or Gott × M against virulent Wa or M virus challenge in gnotobiotic pigs.

| | Primary infection (3-5 days old) | | | | | Challenge (3 weeks after primary infection) | | | | | Median days of | |
|---|---|---|---|---|---|---|---|---|---|---|---|---|
| | Rotavirus administered | | | | | Rotavirus administered | | | | | | Virus |
| | | Serotype/genotype | | | | | Serotype/genotype | | | | | shedding |
| Group (n) | Reassortant | VP7 | VP4 | NSP4 | Titer | Strain | VP7 | VP4 | NSP4 | Titer | Diarrhea | (titer) |
| 1 (4) | Gott × D | G1 | P2B[6] | B | 10⁶ pfu | virulent Wa | G1 | P1A[8] | B | 10⁶ ID₅₀ | | |
| 2 (4) | Gott × D | G1 | P2B[6] | B | 10⁶ pfu | virulent M | G3 | P1A[8] | B | 10⁶ ID₅₀ | | |
| 3 (4) | Gott × M | G3 | P2B[6] | B | 10⁶ pfu | virulent Wa | G1 | P1A[8] | B | 10⁶ ID₅₀ | | |
| 4 (4) | Gott × M | G3 | P2B[6] | B | 10⁶ pfu | virulent M | G3 | P1A[8] | B | 10⁶ ID₅₀ | | |
| 5 (2) | NA | NA | NA | NA | NA | virulent Wa | G1 | P1A[8] | B | 10⁶ ID₅₀ | | |
| 6 (2) | NA | NA | NA | NA | NA | virulent M | G3 | P1A[8] | B | 10⁶ ID₅₀ | | |

After challenge, piglets are examined daily for clinical sings and infection is determined by several criteria, including the onset, duration, and quantity of virus shedding and the onset, duration and severity of diarrhea. Daily rectal swaps/feces can be collected processed and titrated for rotavirus by CCIF assays and ELISA (Yuan et al., J. Virol. 1996, 70:3075-3083; Parreno et al., J. Gen. Virol. 1999, 80:1417-1428).

Sera is collected at inoculation, then weekly, and stored at −20° C. until tested. Samples can be tested for antibody isotype using any standard assay, e.g., ELISA. The virus neutralizing titer in serum is measured using a plaque reduction assay as described above.

### Results

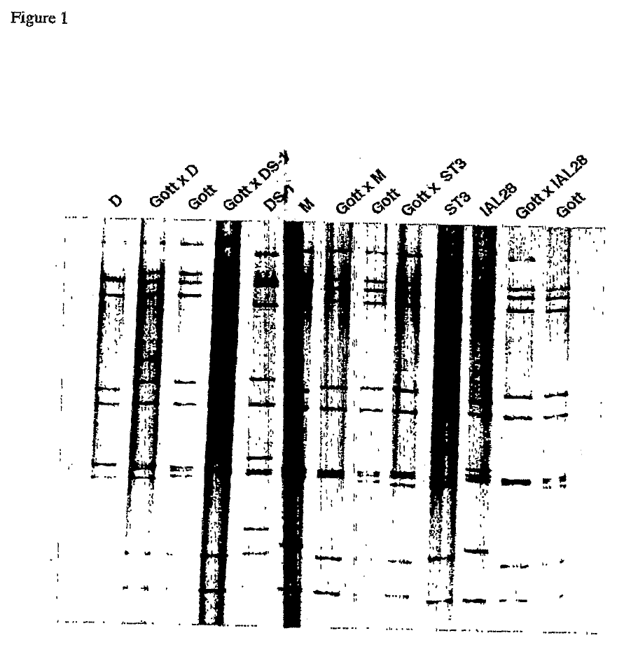

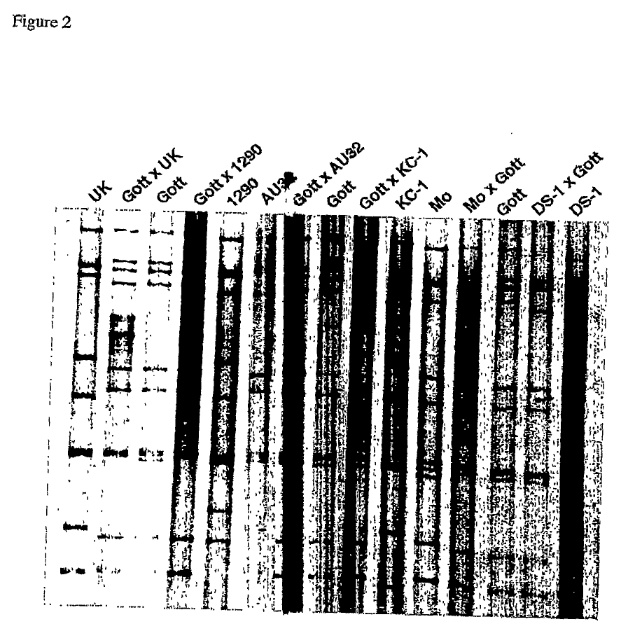

Under the selective pressure of neutralizing monoclonal antibodies ST-2G7, 2C9 or S2-2G10, 72 plaques each were picked and analyzed in order to identify the desired single VP7 or VP4 gene substitution reassortant Gott x D (P2B[6],G1), Gott x DS-1 (P2B[6],G2), Gott x M (P2B[6],G3), Gott x ST3 (P2B[6],G4), Gott x IAL28 (P2B[6],G5), Gott x UK (P2B[6],G6), Gott x 1290 (P2B[6],G8), Gott x AU32 (P2B[6],G9), Gott x KC-1 (P2B[6],G10) or Gott x DS-1 (P1B[4],G4), whereas 144 plaques were analyzed to obtain the desired Gott x Mo (P1A[8],G4) reassortant. As reported previously (Hoshino et al., Vaccine, 2003, 21:3003-3010), seventy-two plaques were routinely picked for initial screening by PAGE analysis since they could be analyzed simultaneously in one PAGE run which involved four gels (18 plaques plus two parental control viruses per gel). Each of the desired reassortants derived a single VP7 gene from D, DS-1, M, ST3, IAL28, UK, 1290, AU32 or KC-1, or a single VP4 gene from Mo or DS-1 and the remaining 10 genes from porcine rotavirus Gottfried as confirmed by PAGE (FIGS. 1 and 2) and CDGE (data not shown) analyses.

Guinea pigs that were hyperimmunized with each of the eleven Gottfried-based vaccine candidates developed high levels of VP7 (1-6, 8-10)-homotypic (range 1:10240 to greater than 1:81920) as well as moderate levels of VP4 (1A, 1B and 2B)-homotypic (range 1:160 to 1:2560) neutralizing antibodies (Table 2). In addition, varying degrees of heterotypic (less than 1:80 to 1:2560) neutralizing antibodies were detected. It was of note that the single VP7 gene substitution reassortant vaccine candidates induced in immunized animals varying degrees (range from less than 1:80 to 1:640) of neutralizing antibodies to selected P1A[8] or P1B[4] viruses which could not be detected previously in animals immunized with simian RRV-based or bovine UK-based single VP7 gene substitution reassortant vaccines (Hoshino et al., Vaccine, 2003, 21:3003-3010).

TABLE 2
Antigenic characterization of Gottfried-based single VP7 or VP4 gene substitution reassortants
by analysis of their guinea pig hyperimmune antiserum neutralization profile.

| Rotavirus tested | | | | | Recipricol of 60% PRN antibody titer of guinea pig antiserum induced | | | | |
|---|---|---|---|---|---|---|---|---|---|
| against serum | | | | | by immunization with indicated VP7 or VP4 reassortant¹ | | | | |
| | | G | P type | | Gott × D | Gott × DS-1 | Gott × M | Gott × ST3 | Gott × IAL28 |
| Strain | Host | type | [genotype] | | P2B[6], G1 | P2B[6], G2 | P2B[6], G3 | P2B[6], G4 | P2B[6], G5 |
| Wa | Human | 1 | 1A | [8] | 20480² | 80 | 160 | 80 | 80 |
| DS-1 | Human | 2 | 1B | [4] | 160 | 10240 | 160 | 80 | <80 |
| P | Human | 3 | 1A | [8] | 160 | 160 | 10240 | 80 | 2560 |
| ST3 | Human | 4 | 2A | [6] | 160 | 320 | 160 | 10240 | 160 |
| Gott | Porcine | 4 | 2B | [6] | 640 | 2560 | 640 | 10240 | 640 |
| IAL28 | Human | 5 | 1A | [8] | 80 | 80 | 160 | 160 | 10240 |
| PA169 | Human | 6 | 11 | [4] | 80 | 80 | 160 | 80 | 160 |
| UK | Bovine | 6 | 7 | [5] | <80 | <80 | <80 | <80 | 80 |
| 69M | Human | 8 | 4 | [10] | <80 | <80 | <80 | <80 | 640 |
| 1290 | Human | 8 | 1B | [4] | 80 | <80 | 80 | <80 | <80 |
| WI61 | Human | 9 | 1A | [8] | 80 | 80 | 80 | 80 | 160 |
| AU32 | Human | 9 | 1A | [8] | 160 | 160 | 160 | 160 | 640 |
| A64 | Human | 10 | [14] | | <80 | <80 | <80 | <80 | <80 |
| KC-1 | Bovine | 10 | [?] | | <80 | <80 | <80 | <80 | <80 |
| L26 | Human | 12 | 1B | [4] | <80 | 80 | 80 | 80 | <80 |

| | Recipricol of 60% PRN antibody titer of guinea pig antiserum induced | | | | | |
|---|---|---|---|---|---|---|
| Rotavirus tested | by immunization with indicated VP7 or VP4 reassortant¹ | | | | | |
| against serum | Gott × UK | Gott × 1290 | Gott × AU32 | Gott × KC-1 | Mo × Gott | DS-1 × Gott |
| Strain | P2B[6], G6 | P2B[6], G8 | P2B[6], G9 | P2B[6], G10 | P1A[8], G4 | P1B[4], G4 |
| Wa | 80 | 160 | 160 | 160 | 640 | 80 |
| DS-1 | 80 | 80 | 80 | <80 | <80 | 1280 |
| P | 80 | 320 | 160 | 80 | 320 | 160 |
| ST3 | 160 | 320 | 160 | 160 | 10240 | 10240 |
| Gott | 640 | 1280 | 640 | 1280 | 81920 | 40960 |
| IAL28 | 80 | 160 | 160 | 160 | 320 | 80 |
| PA169 | 10240 | 80 | 80 | 80 | 80 | 80 |
| UK | 20480 | <80 | <80 | <80 | <80 | <80 |
| 69M | 80 | 40960 | <80 | <80 | 80 | 80 |
| 1290 | 80 | >81920 | 80 | <80 | 80 | 640 |
| WI61 | 80 | 80 | 10240 | 160 | 160 | 80 |
| AU32 | 160 | 160 | 10240 | 640 | 320 | <80 |
| A64 | <80 | <80 | <80 | 10240 | <80 | <80 |
| KC-1 | <80 | <80 | <80 | 20480 | <80 | <80 |
| L26 | <80 | 80 | 80 | <80 | 80 | 640 |

¹The homologous 60% PRN antibody titers ranged from 10240 to 81920 in previous tests in this laboratory
²VP7 homotypic antibody titers are underlined

### Discussion

The advent in 1998 of the first licensed human rotavirus vaccine, a RRV-based quadrivalent vaccine (RotaShield™, Wyeth-Ledarie Vaccines and Pediatrics, Philadelphia, Pa.) (Kapikian, in, Chadwick and Goode, eds, Novartis Foundation Symposium 238, Gastroenteritis Viruses, Chichester, UK, John Wiley & Sons, LTD, pgs. 153-179, 2001), provided an impetus for the expansion of programs of global rotavirus strain surveillance and assessment of the rotavirus disease burden (for reviews, see (Santos et al., Rev. Med. Virol., 2005, 15:29-56); Kirkwood, Expert Opin. Biol. Ther., 2003, 3:97-105; Bresee et al., Emerg. Infect. Dis., 2004, 10:988-995)). In addition, availability in the early 1990s of a reliable and relatively easy methodology (i.e., RT-PCR typing) for rotavirus G and P genotyping (Das et al., J. Clin. Microbiol., 1994, 32:1820-1822; Fischer et al., Rev. Med. Virol., 2004, 14:71-82; Gentsch et al., J. Clin. Microbiol., 1992, 30:1365-1373; Gouvea et al., 1990, J. Clin. Microbiol., 28:276-282) accelerated this trend. Thus, a large number of publications reporting rotavirus strain surveillance data have been published from around the world since the 1990s (for reviews, see (Santos et al., Rev. Med. Virol., 2005, 15:29-56; Cunliffe et al., 2002, Lancet, 359:640-642; Desselberger et al., in, Chadwick and Goode, eds, Novartis Foundation Symposium 238, Gastroenteritis Viruses, Chichester, UK, John Wiley & Sons, LTD, pgs. 125-152, 2001; Gentsch et al., J. Infect. Dis., 1996, 174(Suppl. 1):S30-S36; Koshimura et al., Microbiol. Immunol., 2000, 44:499-510). Such studies have (i) confirmed the epidemiologic importance of four globally common P-G combinations (i.e., P[8],G1; P[4],G2; P[8],G3; and P[8],G4), (ii) demonstrated the global distribution of G9 viruses in conjunction with various P types, (iii) detected unusual G and P types (e.g., G5, G6, G8, G10, G12 and P[6]) in various regions of the world and (iv) identified an enormous variety of P-G combinations in rotavirus field isolates. Such information has indeed influenced the approaches to the development of an effective rotavirus vaccine. For example, additional rhesus rotavirus-based and bovine rotavirus-based candidate vaccines have been constructed (Hoshino et al., Vaccine, 2003, 21:3003-3010) that would give antigenic coverage for G5, G8, G9 and G10 and could be added to the existing candidate vaccine compositions which are designed to cover four globally common G types (i.e., G1, G2, G3 and G4). In the present invention, based on the modified Jennerian approach to immunization, 11 porcine rotavirus Gottfried-based single VP7 or VP4 gene substitution reassortant compositions have been generated which could provide (i) an attenuation phenotype of a porcine rotavirus in humans and (ii) antigenic coverage not only for G1, G2, G3 and G4 but also G5, G6, G8, G9 and G10 as well as P1A[8], P1B[4] and P2A[6]. The Gottfried strain was chosen because the VP4 (P2B[6]) of the Gottfried strain was closely related serotypically to a human rotavirus VP4 which has P2A[6] specificity (Hoshino et al., Virology, 2003, 316:1-8; Gorziglia et al., J. Virol., 1990, 64:414-418; Li et al., J. Clin. Micrbiol., 1993 31:3075-3077). This may give the Gottfried rotavirus-based reassortant compositions an advantage over rhesus RRV-based or bovine UK rotavirus-based reassortant compositions since the RRV VP4 (P5B[3]) or UK VP4 (P7[5]) of such compositions is not related serotypically to the P2A[6] VP4 or to other human rotavirus VP4 serotypes (Hoshino et al., Vaccine, 2002, 20:3576-3584). As stated earlier, the VP4 gene with a P[6] allele in conjunction with various G types has been detected in an increasing number of patients with diarrhea worldwide. For example, approximately 30% of a total of 1407 rotavirus-positive diarrheal stools collected in 8 countries in Africa from 1978 to 2001 were shown to bear the P[6] specificity (Steele et al., Vaccine, 2003, 21:361-367). In addition, recent studies indicate that the contemporary G9 viruses belonging to VP7 phylogenetic lineage 3 have been detected in association with P[6] VP4 in various countries on four of 5 continents (for reviews, see (Santos et al., Rev. Med. Virol., 2005, 15:29-56).

Previously, Kang et al (J. Clin. Microbiol., 1989, 27:2744-2750) analyzed by plaque reduction neutralization assay nine neutralizing monoclonal antibodies (N-mAbs) raised against the VP4 of the Gottfried strain and reported that (i) three N-mAbs neutralized all nine human rotavirus strains tested; four P1A[8] viruses (strains Wa, M, VA70 and WI61), one P1B[4] virus (strain DS-1) and four P2A[6] viruses (strains M37, 1076, McN13 and ST3), (ii) another three N-mAbs neutralized four 1A[8] viruses, one 1B[4] virus and one P2A[6] virus (ST3), (iii) one N-mAb neutralized three P1A[8] viruses (Wa, M and W161) and one P1B[4] virus, (iv) one N-mAb neutralized four P2A[6] viruses only, and (v) one N-mAb did not neutralize any of nine human rotavirus strains except for the homologous porcine Gottfried strain. Of interest was the finding that in that study, various rotavirus strains of animal origin including (i) simian SA11 bearing P5B[2] and rhesus rotavirus RRV bearing P5B[3], (ii) porcine OSU and SB-1A bearing P9[7], and (iii) bovine NCDV bearing P6[1] and B223 bearing P8[11] were not neutralized by any of the nine neutralizing monoclonal antibodies (N-mAbs) tested. Thus, that study demonstrated that certain neutralization epitopes on the Gottfried VP4 (P2B[6]) were shared not only with P2A[6] VP4 but also with P1A[8] and P1B[4] VP4s, which are the predominant rotavirus P types detected in humans with diarrhea globally. Such observations were confirmed and extended in the present study in which guinea pigs hyperimmunized with each of the nine Gottfried-based single VP7 gene substitution reassortant vaccine candidates developed low but consistent levels of antibodies capable of neutralizing selected P1A[8] and to a lesser extend P1B[4] viruses as well. In a previous study, such neutralizing antibodies to P1A[8] or P1B[4] VP4 were not detected in guinea pigs hyperimmunized with rhesus RRV-based or bovine UK-based single VP7 gene substitution reassortant vaccine candidates (Hoshino et al., Vaccine, 2003, 21:3003-3010). In addition, rhesus RRV VP4, bovine UK and WC3 VP4 have been reported not to share any neutralization epitopes with P1A[8], P1B[4] or P2A[6] VP4 (Hoshino et al., Vaccine, 2002, 20:3576-3584). Thus, the VP4 of the Gottfried-based vaccines may provide an additional advantage over rhesus-based or bovine-based vaccines. The evaluation of the selected Gottfried-based vaccine candidates in a gnotobiotic piglet model for their immunogenicity and associated efficacy will be of interest since (i) the disclosed Gottfried-human rotavirus reassortants would replicate in gnotobiotic piglets and (ii) selected diarrheagenic human rotaviruses in piglets (e.g., strain Wa with a P1A[8],G1 specificity and strain M with a P1A[8],G3 specificity) are available for challenge studies (Chang et al., Virus Genes, 1999, 18:229-233).

A RRV-based quadrivalent vaccine (RotaShield™) consists of four components: three RRV-human reassortants representing G1, G2 and G4 plus RRV itself representing G3 (Advisory Committee on immunization Practices, MMWR Morb. Mortal. Weekly Rep., 1999, 53:949-954). It is of interest to note that vaccine viruses recovered from infant vaccinees who were given the RRV-based quadrivalent vaccine were reported to be predominantly G3 component RRV (Kobayashi et al., J. Infect. Dis., 1994, 170:1260-1263; Ward et al., Pediatr. Infect. Dis. J., 1998, 17:386-390). In addition, horizontal transmission of vaccine strains observed in the RRV-based quadrivalent vaccine field trials was associated predominantly with the G3 component in a Venezuelan trial (Hoshino et al., J. Infect. Dis., 2003, 187:791-800) and exclusively with RRV in a Bangladesh trial (Bresee et al., Pediatr. Infect. Dis. J., 2001, 20:1136-1143). These findings appear to suggest that the RRV (which bears all 11 RRV genes) has an advantage over the other three RRV-human reassortants (each of which bears 10 RRV genes and one human rotavirus VP7 gene) in terms of its replication capability in human intestine. It has been previously reported that the porcine Gottfried strain was similar, if not identical, serotypically to human rotavirus stain ST4 (G4) (Hoshino et al., J. Infect. Dis., 1984, 149:694-702) which is identical to strain ST3 (G4) as determined by neutralization and RNA-RNA hybridization (Hoshino et al., J Clin Microbiol, 1985, 21:425-430). Thus, in the present invention, instead of choosing the Gottfried (P2B[6],G4) itself as a G4 component, a reassortant Gott x ST3 (P2B[6],G4) has been generated). Hence, each of the 11 Gottfried-based candidate immunogenic compositions constructed in the present invention, which bear 10 porcine rotavirus Gottfried genes and only one human rotavirus VP7 or VP4 gene (or a gene which encodes a protein immunologically cross-reactive with human VP7), may exhibit a similar level of replication capability in the human intestine.

In summary, eleven porcine rotavirus Gottfried-based reassortants have been constructed which could provide (i) an attenuation phenotype of a porcine rotavirus in humans and (ii) antigenic coverage for G serotypes 1, 2, 3, 4, 5, 6, 8, 9 and 10 and P serotypes P1A[8], P1B[4] and P2A[6]. Such compositions can be used singly or in combinations of two or more depending upon the regional requirement warranted from epidemiologic studies.

### Microorganism Deposit Information

The human rotavirus strains were deposited with the American Type Culture Collection, 10801 University Boulevard, Manassas, Va. 20110-2209, Jul. 8, 2005, under the conditions of the Budapest Treaty and designated as follows.

| Reassortant Designation | ATCC Accession Number |
|---|---|
| Gott x D (P2B[6], G1) | ATCC PTA-6840 |
| Gott x DS-1 (P2B[6], G2) | ATCC PTA-6841 |
| Gott x M (P2B[6], G3) | ATCC PTA-6842 |
| Gott x ST3 (P2B[6], G4) | ATCC PTA-6843 |
| Gott x IAL28 (P2B[6], G5) | ATCC PTA-6844 |
| Gott x UK (P2B[6], G6) | ATCC PTA-6845 |
| Gott x 1290 (P2B[6], G8) | ATCC PTA-6846 |
| Gott x AU32 (P2B[6], G9) | ATCC PTA-6847 |
| Gott x KC-1 (P2B[6], G10) | ATCC PTA-6848 |
| Mo x Gott (P1A[8], G4) | ATCC PTA-6849 |
| DS-1 x Gott (P1B[4], G4) | ATCC PTA-6850 |

Although the foregoing invention has been described in some detail by way of illustration and example for purposes of clarity of understanding, it will be obvious that certain changes and modifications may be practiced within the scope of the appended claims. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted.

What is claimed is:

1. An immunogenic composition comprising at least one rotavirus reassortant, wherein each rotavirus reassortant comprises at least one gene encoding a protein immunologically cross-reactive with each of a human VP7 serotype G1, VP7 serotype G2, VP7 serotype G3, VP7 serotype G4, VP7 serotype G5, VP7 serotype G6, VP7 serotype G8, VP7 serotype G9, VP7 serotype G10, VP4 serotype P1A, VP4 serotype P1B and VP4 serotype P2A, and at least one gene which encodes at least one of the remaining porcine Gottfried strain rotavirus proteins, wherein the composition induces an immune response capable of reducing the clinical symptoms associated with gastroenteritis caused by rotavirus infection.

2. The immunogenic composition according to claim 1, wherein the composition comprises reassortant rotavirus having 10 genes from the porcine rotavirus Gottfried strain and 1 gene from a human rotavirus encoding an antigen having a VP7 serotype G1, VP7 serotype G2, VP7 serotype G3, VP7 serotype G4, VP7 serotype G5, VP7 serotype G8, VP7 serotype G9, VP4 serotype P1A[8], and/or VP4 serotype P1B[4].

3. The immunogenic composition according to claim 2, wherein the composition comprises reassortant rotavirus having 10 genes from the porcine rotavirus Gottfried strain and 1 gene from an animal rotavirus which encodes an antigen immunologically cross-reactive with an antigen having human VP7 serotype 10 or human VP7 serotype 6.

4. The immunogenic composition according to claim 2, wherein the composition comprises porcine x human reassortant rotavirus encoding an antigen having a human VP7 serotype G1, human VP7 serotype G2, human VP7 serotype G3, and human VP7 serotype G4.

5. The immunogenic composition according to claim 4, wherein the composition further comprises porcine x human reassortant rotavirus encoding an antigen having a human VP7 serotype G5, G8, G9, and/or a human VP4 serotype P1A[8], and/or P1B[4].

6. The immunogenic composition according to claim 4, wherein the composition further comprises porcine x human reassortant rotavirus encoding an antigen having a human VP4 serotype P1A[8], and/or P1B[4].

7. The immunogenic composition according to claim 1, wherein the composition comprises at least one of the porcine reassortant rotavirus Gott x D (P2B[6],G1), Gott x DS-1 (P2B[6],G2), Gott x M (P2B[6],G3), Gott x IAL28 (P2B[6],G5), Gott x UI (P2B[6],G6), Gott x 1290 (P2B[6],G8), Gott x AU32 (P2B[6],G9), Gott x KC-1 (P2B[6],G10), Mo x Gott (P1A[8],G4) or DS-1 x Gott (P1B[4],G4).

8. The composition according to claim 7, wherein the composition comprises the porcine x human reassortants Gott x D, Gott x DS-1, Gott x P, Gott x ST-3, Gott x 1290 and Gott x AU32.

9. The composition according to claim 8, wherein each human x bovine rotavirus reassortant is present at an amount of less than 10⁶ plaque forming units (pfu) for each dosage to be administered.

10. The composition according to claim 5, wherein the composition comprises the porcine x human reassortants Gott x D, Gott x DS-1, Gott x P, Gott x ST-3, Gott x 1290, Gott x AU32, and Gott x IAL28.

11. The composition according to claim 10, wherein each porcine x human rotavirus reassortant is present at an amount of less than 10⁶ pfu for each dosage to be administered.

12. The composition according to claim 3, wherein each porcine rotavirus reassortant comprises porcine x human reassortants Gott x D, Gott x DS-1, Gott x P, Gott x ST-3, Gott x 1290, Gott x AU32, and porcine x bovine reassortants Gott x KC-1 and Gott x UK.

13. The composition according to claim 12, wherein each porcine rotavirus reassortant is present at an amount of less than 10⁶ pfu for each dosage to be administered.

14. The compositions according to claims 1 through 13 further comprising a porcine x human reassortant rotavirus of VP4 serotype P1A[8] and/or VP4 serotype P1B[4].

15. The composition according to claim 1 further comprising a physiologically acceptable carrier.

16. The composition according to claim 15, wherein the composition further comprises an adjuvant.

17. The composition according to claim 1, wherein the composition is lyophilized.

18. The composition according to claim 3, wherein each porcine reassortant rotavirus is formulated to provide a dosage of 10³ to 10⁸ pfu for each dosage administered.

19. The composition according to claim 18, wherein each porcine reassortant rotavirus is formulated to provide a dosage of 10³ to less than 10⁶ pfu for each dosage administered.

20. A method for stimulating the immune system to produce an immunogenic response to human rotavirus VP7 and/or VP4 serotype antigen which method comprises administering a multivalent immunogenic composition comprising at least one rotavirus reassortants, wherein the rotavirus reassortant comprises at least one gene encoding a protein immunologically cross-reactive with each of a human VP7 serotype G1, VP7 serotype G2, VP7 serotype G3, VP7 serotype G4, VP7 serotype G5, VP7 serotype G6, VP7 serotype G8, VP7 serotype G9, VP7 serotype G10, VP4 serotype P1A, VP4 serotype P1B and VP4 serotype P2A, and at least one gene which encodes at least one of the remaining porcine Gottfried strain rotavirus proteins, and a pharmaceutically acceptable carrier.

21. The method according to claim 20, wherein the porcine x human rotavirus reassortant comprises the porcine rotavirus reassortant encoding the human VP7 serotype 1, the human VP7 serotype 2, the human VP7 serotype 3, the human VP7 serotype 4, and the human VP7 serotype 8, the human VP7 serotype 9, the human VP4 serotype P1A[8] and/or the human VP4 serotype P1B[4].

22. The method according to claim 20, wherein the reassortant porcine rotavirus comprises the porcine x human rotavirus reassortant encoding the human VP7 serotype 1, the human VP7 serotype 2, the human VP7 serotype 3, the human VP7 serotype 4, and the human VP7 serotype 8, the human VP7 serotype 9, the human VP4 serotype P1A[8], the human VP4 serotype P1B[4] and/or the porcine x bovine rotavirus reassortant encoding a antigen immunologically cross-reactive with the human VP7 serotype 10.
